(19) United States
(12) Patent Application Publication
(10) Pub. No.: US 2008/0306429 A1
(43) Pub. Date: Dec. 11, 2008

(54) UVEOSCLERAL DRAINAGE DEVICE

(76) Inventors: Milton B. Shields, Hamden, CT (US); Ben Bronstein, Newton, MA (US); Nicholas Fish Warner, Cummington, MA (US)

Correspondence Address: PEPPER HAMILTON LLP, ONE MELLON CENTER, 50TH FLOOR, 500 GRANT STREET, PITTSBURGH, PA, 15219, US

(21) Appl. No.: 12/135,848

(22) Filed: Jun. 9, 2008

Related U.S. Application Data
(60) Provisional application No. 60/942,622, filed on Jun. 7, 2007.
(60) Provisional application No. 60/954,258, filed on Aug. 6, 2007.

(51) Int. Cl. A61M 1/00

(52) U.S. Cl. 604/8

(57) ABSTRACT

An ophthalmic shunt implantable in an eye has an elongate body and a conduit for conducting aqueous humor from an anterior chamber of the eye to the suprachoroidal space of the eye. The elongate body has a forward end, a spaced back end, and an insertion head that extends from the forward end. The insertion head defines a shearing edge suitable for cutting eye tissue engage thereby. The elongate body can define at least one slot that is configured for operative receipt of a surgical tool such as an obturator. In another aspect, at least a portion of the conduit can be configured for operative receipt of a surgical tool such as an obturator.

### CROSS-REFERENCE TO RELATED APPLICATIONS

This application claims the priority benefit of U.S. Provisional Patent Application No. 60/942,622 filed Jun. 7, 2007 and U.S. Provisional Patent Application No. 60/954,258 filed Aug. 6, 2007, the subject matter of which are both incorporated herein by reference.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

The invention generally relates to eye implants, more particularly, to an ophthalmic shunt, an ophthalmic shunt assembly and method of using same for use in enhancing uveoscleral drainage in the eye to lower eye pressure.

Glaucoma, a leading cause of world blindness, is a group of disorders, characterized by irreversible damage to the optic nerve, or glaucomatous optic neuropathy, in which elevated intraocular pressure is the main causative risk factor. A proven way to prevent the blindness of glaucoma is to control the intraocular pressure.

Clinical management of intraocular pressure can be achieved medically or surgically. Modern medical therapy for glaucoma began in the 1870s, with the introduction of pilocarpine and other cholinergic agonists. In the twentieth century, several compounds were introduced, such as alpha-2 agonists, beta-adrenergic antagonists, topical and systemic carbonic anhydrase inhibitors, and prostaglandins. However, glaucoma medication is not available or practical in many parts of the world, and are inadequate in many patients, despite availability. Hence the need for surgical methods to control the intraocular pressure.

Control of intraocular pressure can be achieved surgically by reducing the production of aqueous humor or by increasing its outflow. Operations to reduce production, referred to collectively as cyclodestructive surgery, destroy a portion of the ciliary body, the source of aqueous humor. Destructive elements over the years have included diathermy, cryotherapy and, most recently, laser energy. While these operations are effective in lowering the intraocular pressure, and are beneficial in certain situations, they have a high complication rate, including inflammation and further reduction in visual acuity.

Referring to FIG. 1, after production by the ciliary body, aqueous humor leaves the eye by many routes. Some goes posteriorly through the vitreous body to the retina, while most circulates in the anterior segment of the eye, nourishing avascular structures such as the lens and cornea, before outflow by two main routes: canalicular or uveoscleral.

The canalicular, also referred to as the trabecular or conventional, route is the main mechanism of outflow, accounting for approximately 80% of aqueous egress from the normal eye. The route is from the anterior chamber angle (formed by the iris and cornea), through the trabecular meshwork, into Schlemm's canal. The latter is a 360° channel just peripheral to meshwork. It is connected to intrascleral outlet channels that take the aqueous through the sclera to reunite with the blood stream in the episcleral veins.

The uveoscleral route is less clear with regard to anatomy and physiologic significance, but probably accounts for 10-20% of aqueous outflow in the normal human eye. As with the canalicular route, the uveoscleral pathway begins in the anterior chamber angle. The aqueous is absorbed by portions of the peripheral iris, the ciliary body and probably the trabecular meshwork, from whence it passes posteriorly through the longitudinal muscle of the ciliary body to the suprachoroidal space (between the choroids and sclera). Aqueous in the suprachoroidal space may pass as far posteriorly as the optic nerve and leave the eye through a variety of emissaria around nerves and vessels in the sclera.

The ideal glaucoma operation would be to re-establish normal canalicular flow into Schlemm's canal. In some forms of glaucoma this is possible, such as the iridectomy (introduced in the 1850s) for pupillary block glaucoma and goniotomy and trabeculotomy (introduced in the mid-twentieth century) for congenital glaucoma. For the vast majority of glaucomas, however, the obstruction to outflow (and, hence, the elevated intraocular pressure) is in the trabecular meshwork, and the only effective surgical approach has been to bypass the normal canalicular pathway and create bulk outflow by one of two methods: filtration surgery and drainage implant devices.

Filtration surgery was introduced in the first decade of the twentieth century. The basic principle is the creation of a fistula through trabecular meshwork, Schlemm's canal and sclera. Aqueous flows through the fistula to create a pool beneath the elevated conjunction (called a bleb), through which it filters to wash away in the tear film. The basic operation, in a variety of modified forms, has now been the preferred glaucoma procedure for nearly 100 years, despite serious limitations.

Limitations of filtering surgery include failure due to fibrotic closure of the fistula. Of even greater concern are the complications associated with excessive outflow, which include an intraocular pressure that is too low (hypotony) and a conjunctival filtering bleb that becomes too thin, with leakage and the risk of infection (endophthalmitis).

Drainage implant surgery was developed primarily to overcome the problem of fistula closure, since a conduit passes from the anterior chamber angle, through the fistula, to a plate beneath the conjuctiva. However, these operations are also complicated by early hypotony and late failure due to obstruction of the conduit or excessive fibrosis over the plate. There is a need, therefore, for a device and method of using same that reliably channels aqueous into pathways without creating hypotony or a filtering bleb.

Although the uveoscleral pathway may only account for 10-20% of aqueous outflow in the normal state, there is evidence that it can be enhanced to accommodate a significantly greater percentage of outflow. For example, topical prostaglandins, which work nearly exclusively by increasing uveoscleral outflow, can lower the intraocular pressure by 30-50% in some patients. Even more compelling are the results of early surgical attempts to enhance uveoscleral outflow.

In the first decade of the twentieth century, paralleling the introduction of filtering surgery, an operation was devised to enhance uveoscleral outflow, called cyclodialysis. Referring to FIGS. 2A and 2B, the basic principle is separation of the ciliary body from the scleral spur, which provides a direct route for aqueous flow from the anterior chamber angle to the suprachoroidal space. Unlike filtering surgery, however, cyclodialysis enjoyed only limited acceptance in the twentieth century. Although it was commonly used during the first half of the century, serious limitations led to its virtual abandonment by mid-century. The limitations were two-fold. When so-called cyclodialysis cleft was patent, the operation often worked too well, with significant hypotony. In many patients, the cleft would close suddenly, with a profound rise in the intraocular pressure.

A variety of efforts have been made to prevent closure of the cleft by wedging flaps of ocular tissue or plastic devices into the space. To date, none of these techniques have proved successful.

### SUMMARY

The present invention relates to eye implant devices for lowering intraocular pressure in an eye. In one example, an ophthalmic shunt suitable for implantation in an eye is provided. In this example, the shunt may have an elongate body and a conduit for conducting aqueous humor from an anterior chamber of the eye to the suprachoroidal space of the eye. The elongate body may have a forward end, a back end, and an insertion head that extends from the forward end. The insertion head may include a shearing edge suitable for cutting eye tissue engaged thereby. Together, the forward end and the insertion head of the body may include a shoulder surface suitable for sealing any incision created in the eye tissue.

In one example, the elongate body may have a substantially fusiform cross-sectional shape on at least a portion of its elongate length. The elongate body may also have an arcuate shape along at least a portion of its length with a radius of curvature suitable for extending along the curvature of the sclera of the eye. In another aspect, at least a portion of the elongate body can have an arcuate cross-sectional shape along at least a portion of the length of the elongated body with a radius of curvature suitable for extending along the curvature of the sclera of the eye.

The conduit of the shunt may have a first end defined within a portion of a top surface of the insertion head and a second end defined within a portion of the back end of the elongate body. In one exemplary aspect, the first end of the conduit may be positioned at the shearing edge of the insertion head. In some embodiments, the conduit may include one or more conduits.

In another embodiment, at least a portion of the conduit may be configured to receive a surgical tool or an obturator. In one exemplary aspect, an obturator may be provided that has a portion that is configured for integral use with the ophthalmic shunt. In this example, the obturator includes a handle and a mount portion. The handle may have a proximal end portion that extends along a longitudinal axis and a distal end portion that is oriented at an angle relative to the longitudinal axis of the proximal end portion. In one embodiment, the mount portion may have a first end and a second end. In this embodiment, the first end of the mount portion may connect to the distal end portion of the handle and extends outwardly to the second end of the mount portion, and at least a portion of the second end may be configured to be received by at least a portion of the conduit.

In an alternative embodiment of the obturator, the mount portion may have an end portion including at least one prong. In this embodiment, the end portion may be connected to the distal end portion of the handle, and the at least one prong may extend outwardly from the end of the mount portion, with the at least one prong being configured to be received by a slot on the ophthalmic shunt.

The shunt may be readily implanted within the eye of a patient in order to reduce the intraocular pressure within the eye. In one example, a first incision in and through the conjunctiva and the sclera at a position posterior to the limbus can be made. The surgeon may then mount the shunt onto an obturator (or alternatively, the shunt can come premounted on the obturator), whereupon the insertion head of the shunt may be passed through the first incision into the supraciliary space of the eye. Next, at least a portion of the shearing edge of the insertion head may be inserted into and through the anterior chamber angle into the anterior chamber of the eye. When the insertion head is inserted within the anterior chamber, the tissue may be stretched and dilated by the shape of the insertion head so that the insertion head is substantially self-sealing. Further, the first end of the conduit may be positioned in fluid communication with the anterior chamber and the second end of the conduit may be placed in fluid communication with the suprachoroidal space when the insertion head is inserted within the anterior chamber. Following removal of the obturator, aqueous humor may be allowed to flow from the anterior chamber of the eye to the suprachoroidal space, which allows the intraocular pressure in the eye to be lowered.

In one aspect, the removal of the obturator can act to prime the conduit. That is, as the obturator is removed from the conduit, it may aspirate fluid into the conduit while displacing air.

In use, the shunt may prevent cleft closure and control the rate of aqueous flow into the suprachoroidal space via the conduit. Thus, the design of the present invention overcomes the limitations inherent in the traditional cyclodialysis procedure: hypotony and cleft closure.

Thus, various embodiments, of the invention are directed to an ophthalmic shunt implantable in an eye including an elongate body having a forward end, a back end, and a tapered insertion head extending from the forward end of the elongate body, the insertion head defining a shearing edge constructed and arranged for cutting eye tissue engaged thereby, the forward end and the insertion head of said body further defining a shoulder surface and a conduit having first end defined at the shearing edge of said insertion head and extending through said body from the forward end to the back end thereof. In some embodiments, the elongate body may be configured to position at least a portion of the insertion head and the first end of the conduit through an incision formed by the shearing edge of the insertion head and into fluid communication with the anterior chamber of the eye and to seat at least a portion of the insertion head against the incision.

In some embodiments, the elongate body may have a substantially fusiform cross-sectional shape. In other embodiments, the elongate body may have a lower surface and a portion of the insertion head may be substantially co-planar to the lower surface thereof. In still other embodiments, the elongate body may have an arcuate shape along at least a portion of its length that is adapted to extend along the curvature of the sclera.

In certain embodiments, the conduit may be configured to receive at least a portion of an obtruator, and in first end of the obturator is at least flush with the first end of the conduit, and in some embodiments, the first end of the conduit may be positioned at an acute angle with respect to the insertion head.

In particular embodiments, the elongate body may have at least one suture hole configured to facilitate suturing the elongate body to eye tissue, and in some embodiments, the elongate body may have a first elongate edge and a spaced second elongate edge wherein said body has at least a pair of spaced suture holes configured to facilitate suturing the elongate body to eye tissue, one suture hole of the pair of spaced suture holes being defined in each respective elongate edge. In other embodiments, the elongate body may have an upper surface and a spaced lower surface wherein the body has at least a pair of spaced suture holes extending between the upper and lower surfaces of said elongate body, the pair of spaced suture holes configured to facilitate suturing the elongate body to eye tissue.

Some embodiments of the invention are directed to an ophthalmic shunt assembly including an elongate body having a forward end, a back end, and an insertion head extending from the forward end of the elongate body, the insertion head having a top surface and defining a shearing edge constructed and arranged for cutting eye tissue engaged thereby, the forward end and the insertion head of said body further defining a shoulder surface a conduit having a first end defined within a portion of said insertion head and extending through said body from the forward end to the back end thereof, the first end at the shearing edge, and an obturator. In certain embodiments, the conduit may be configured to receive at least a portion of the obturator, and the elongate body may be configured to position at least a portion of the insertion head and the first end of the conduit through an incision formed by the shearing edge of the insertion head and into fluid communication with the anterior chamber of the eye and to seat at least a portion of the insertion head against the incision. In such embodiments, the assembly may deliver at least one therapeutic agent to a suprachoroidal space.

In some embodiments, the elongate body has a substantially fusiform cross-sectional shape, and other embodiments, a first end of the obturator may be at least flush with the first end of the conduit. In particular embodiments, the obturator may include a handle located at an opposing end of the first end of the obturator.

In certain embodiments, the elongate body may have at least one suture hole configured to facilitate suturing the elongate body to eye tissue, and in other embodiments, the elongate body may have a first elongate edge and a spaced second elongate edge, and wherein said body has at least a pair of suture holes configured to facilitate suturing the elongate body to eye tissue, one suture hole of the pair of suture holes being defined in each respective elongate edge.

In some embodiments, the conduit may include a valve. In other embodiments, a first end of the conduit may be positioned at an acute angle with respect to the top surface of the insertion head. In still other embodiments, the conduit may include at least a rigid front end, and in further embodiments, the elongate body may include a flexible tube in fluid communication with the conduit to provide a channel for fluid flow from an anterior space to a suprachoroidal space. In certain embodiments, a posterior end of the conduit may include at least one of a plurality of filaments, wires or hollow structures.

Other embodiments of the invention are directed to a method for treating glaucoma in an eye including the steps of providing a biocompatible ophthalmic shunt, wherein the ophthalmic shunt that includes an elongate body having a forward end, a back end, and a tapered insertion head extending from the forward end of the elongate body, the insertion head having a top surface and defining a shearing edge constructed and arranged for cutting eye tissue engaged thereby, the forward end and the insertion head of said body further defining a shoulder surface and a conduit having a first end defined within a portion of said insertion head and extending through said body from the forward end to the back end thereof, the first end at the shearing edge wherein the elongate body is configured to position at least a portion of the insertion head and the first end of the conduit through an incision formed by the shearing edge of the insertion head and into fluid communication with the anterior chamber of the eye and to seat at least a portion of the insertion head against the incision to seal the incision; inserting at least a portion of the shearing edge of the insertion head of the shunt into and through an anterior chamber angle and into the anterior chamber of the eye, with the first end of the conduit into fluid communication with the anterior chamber of the eye; introducing the insertion head anteriorally to seat the shoulder surface of the implant adjacent an interior surface of a supraciliary space of the eye; disposing the back end of the elongate body of the shunt into a suprachoroidal space of the eye so that a second end of the conduit is in fluid communication with the suprachoroidal space; and securing the shunt to the eye.

In some embodiments, the method may further include the step of prior to the insertion of the insertion head into the anterior chamber making a first incision in and through the conjunctiva and the sclera at a position posterior to the limbus, and in certin embodiments, the method may include the step of delivering at least one therapeutic agent to a suprachoroidal space.

### DESCRIPTION OF THE DRAWINGS

For a fuller understanding of the nature and advantages of the present invention, reference should be made to the following detailed description taken in connection with the accompanying drawings, in which:

FIG. 1 is a partial cross-sectional view of an eye showing the normal aqueous flow of aqueous humor though the anterior chamber of the eye.

FIGS. 2A and 2B are partial top views of an eye showing the prior art cyclodialysis operation and the typical result.

FIG. 3A is a perspective view of a first embodiment of the present invention.

FIG. 3B is a perspective view of the embodiment shown in FIG. 3A being grasped by a surgical tool.

FIG. 3C is a cross-sectional view of the embodiment shown in FIG. 3A taken along line 3A.

FIG. 4A is a perspective view of an elongate body of a second embodiment of the present invention.

FIG. 4B is a perspective view of an elongate conduit of the second embodiment of the present invention.

FIG. 4C is a perspective view of the second embodiment with the elongate conduit shown in FIG. 4B disposed within a portion of the elongate body and overlying a portion of a top surface of an insertion head.

FIG. 4D is a perspective view of the second embodiment shown in FIG. 4C being grasped by a surgical tool.

FIG. 5A is a perspective view of an elongate body of a third embodiment of the present invention.

FIG. 5B is a perspective view of an elongate wicking member having an inlet end and an outlet end.

FIG. 5C is a perspective view of the third embodiment with the elongate wicking member shown in FIG. 5B disposed within a slit of the elongate body and overlying a portion of a top surface of an insertion head.

FIG. 5D is a perspective view of the third embodiment of FIG. 5C being grasped by a surgical tool.

FIG. 6A is a partial top view of an eye having an implant, according to the present invention, being positioned into the anterior chamber of the eye.

FIG. 6B is an enlarged cross-sectional detail view of the implant of FIG. 6A.

FIG. 7A is a partial top view of an eye in which an implant according to the present invention is located therein postoperatively.

FIG. 7B is an enlarged cross-sectional detail view of the implant of FIG. 7A.

FIG. 8 is a perspective view of an alternative embodiment of a biocompatible ophthalmic shunt of the present invention shown mounted on a mounting portion of an embodiment of a obturator of the present invention.

FIG. 9 is an enlarged, partially transparent, perspective view of the shunt of FIG. 8 shown mounted thereon the mounting portion of the obturator of FIG. 8.

FIG. 10 is an enlarged perspective view of the shunt of FIG. 8 shown mounted thereon the mounting portion of the obturator of FIG. 8.

FIG. 11 is an enlarged side elevational view of the shunt of FIG. 8 shown mounted thereon the mounting portion of the obturator of FIG. 8.

FIG. 12 is a perspective view of the embodiment of the obturator of FIG. 8.

FIG. 13 is an enlarged perspective view of the obturator of FIG. 12.

FIG. 14 is a side elevational view of the obturator of FIG. 12.

FIG. 15 is an enlarged side elevational view of the obturator of FIG. 12.

FIG. 16 is a top perspective view of the biocompatible ophthalmic shunt of FIG. 8.

FIG. 17 is a bottom perspective view of the shunt of FIG. 16.

FIG. 18 is a side elevational view of the shunt of FIG. 16.

FIG. 19 front end elevational view of the shunt of FIG. 16.

FIG. 20 is a top, partially transparent, plan view of the shunt of FIG. 16, showing an internal conduit.

FIG. 21 is a cross-sectional view of the shunt, taken across line 21-21 of FIG. 20.

FIG. 22 is a cross-sectional view of the shunt, taken across line 22-22 of FIG. 20.

FIG. 23 is a cross-sectional view of the shunt, taken across line 23-23 of FIG. 20.

FIG. 24 is a cross-sectional view of the shunt, taken across line 24-24 of FIG. 10.

FIG. 25 is a side elevational view of the cross-sectional view of the embodiment shown in FIG. 24.

FIG. 26 is a perspective view of an alternative embodiment of a shunt of a present invention, showing a plurality of slots defined therein the body of the shunt.

FIG. 27 is a rear elevational view of the shunt of FIG. 26, showing the plurality of slots and the conduit end.

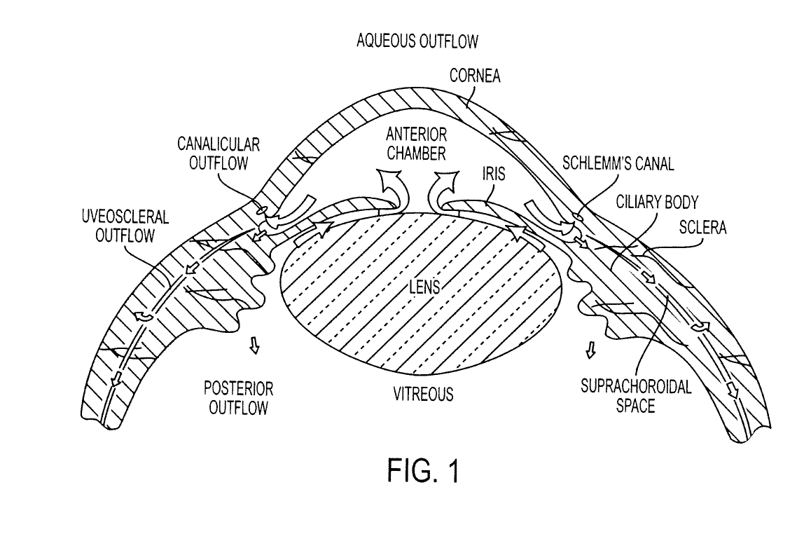

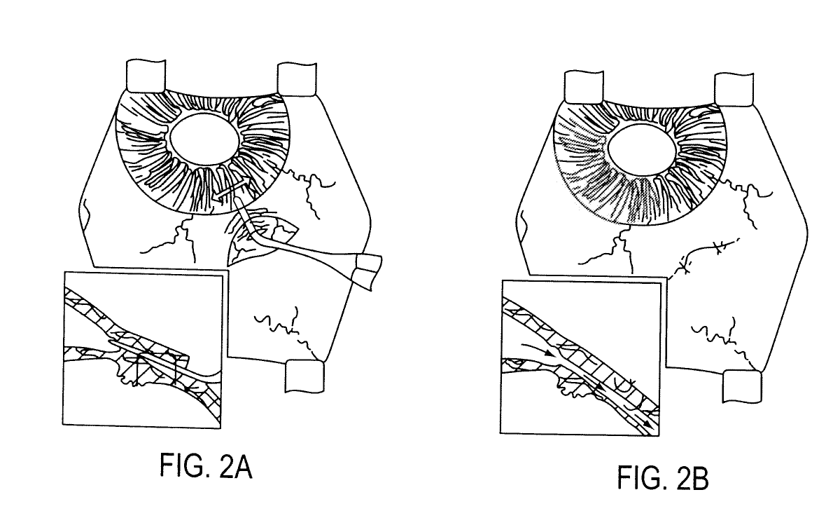

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must be noted that, as used herein, and in the appended claims, the singular forms “a”, “an” and “the” include plural reference unless the context clearly dictates otherwise. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

The invention generally relates to eye implants, more particularly, to an ophthalmic shunt, an ophthalmic shunt assembly and method of using same for use in enhancing uveoscleral drainage in the eye to lower eye pressure.

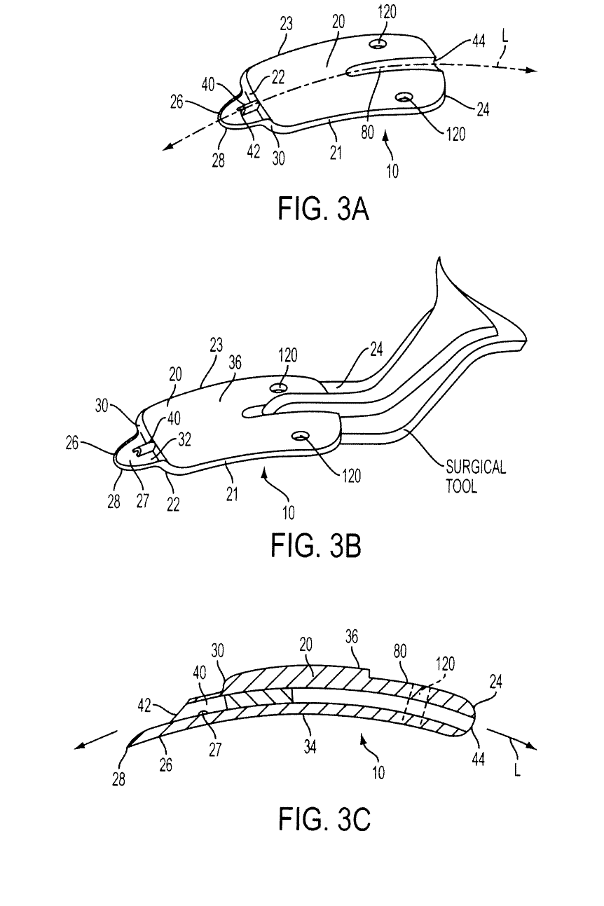

Referring to FIGS. 3A-5D, various exemplary embodiments of uveoscleral drainage devices are shown. The implant or shunt 10 generally includes an uveoscleral drainage device that is adapted for implantation within an eye of a patient. Referring initially to FIGS. 3A-3C, the shunt 10 may include an elongate body 20 and a conduit 40. The elongate body may have a forward end 22, a spaced back end 24, and may extend along a longitudinal axis L. The elongate body 20 may further include a first elongate edge 21 and a second elongate edge 23 that extend respectively from the forward end to the back end of the body. The body may also include an insertion head 26 extending generally longitudinally from the forward end 22. The insertion head 26 may be adapted for insertion into the anterior chamber of the eye and may include a shearing edge 28 constructed and arranged for cutting eye tissue engaged thereby. In some embodiments, the shearing edge 28 of the insertion head 26 may be rounded or arced in shape as shown. However, as one skilled in the art will appreciate, other shapes, such as, for example, chisel shapes, scalpel shapes, and the like, are contemplated and may be used for the shearing edge.

In various embodiments, the junction of the insertion head 26 against the forward end 22 of the elongated body 20 may define a shoulder surface 30. In one embodiment, the insertion head 26 may have a base portion 32 having a first width and where the respective first and second elongate edges are spaced apart and a second width that is greater than the first width. Thus, the insertion head may be tapered such that the width and/or thickness increases from the shearing edge to the junction with the shoulder surface, and in certain embodiments, the width and/or height of the insertion head at the junction with the shoulder may be substantially equal to the width and/or height of the elongated body 20. Without wishing to be bound by theory, the taper of the insertion head may allow the insertion head to seal the incision made by the shearing edge between the anterior chamber and the suprachoroidal space.

The shoulder surface 30 of the body 20 may be adapted to engage tissue portions separating the anterior chamber and the suprachoroidal space. The shoulder surface 30 may also aid in limiting the anterior movement or displacement of the device when implanted, which may help prevent the forward end 22 of the drainage device from penetrating and entering the anterior chamber. In the exemplary embodiment shown, the base portion 32 of the insertion head 26 may extend in a substantially co-planar manner to a lower surface 34 of the elongate body. Alternatively, the insertion head 26 may extend from a portion of the forward end 22 that is spaced from a circumferential edge of the forward end. In this example, the shoulder surface 30 would extend about the periphery of the base portion 32 of the insertion head 26.

The elongated body 20 may have a length from the forward end 22 to the back end 24 extending from proximate the interior surface of the anterior chamber to the suprachoroidal space of the eye. The back end 24 of the body 20 may be adapted for insertion within the suprachoroidal space of the eye. Along at least a portion of its length, the body may be substantially planar or may have an arcuate shape that is adapted to extend along a portion of the curvature of the sclera of the eye. As one will appreciate from the illustrated embodiment, the body may be thin to provide a less irritating fit within the eye.

In some exemplary embodiments, the elongate body 20 may have a substantially fusiform cross-sectional shape. Without wishing to be bound by theory, this substantially fusiform shape may aid in stabilizing the device when implanted as tissues of the anterior chamber angle surround portions of the exterior surface of the body. A variety of cross-sectional shapes are contemplated for the elongate body as long as a shoulder surface is defined in the forward end.

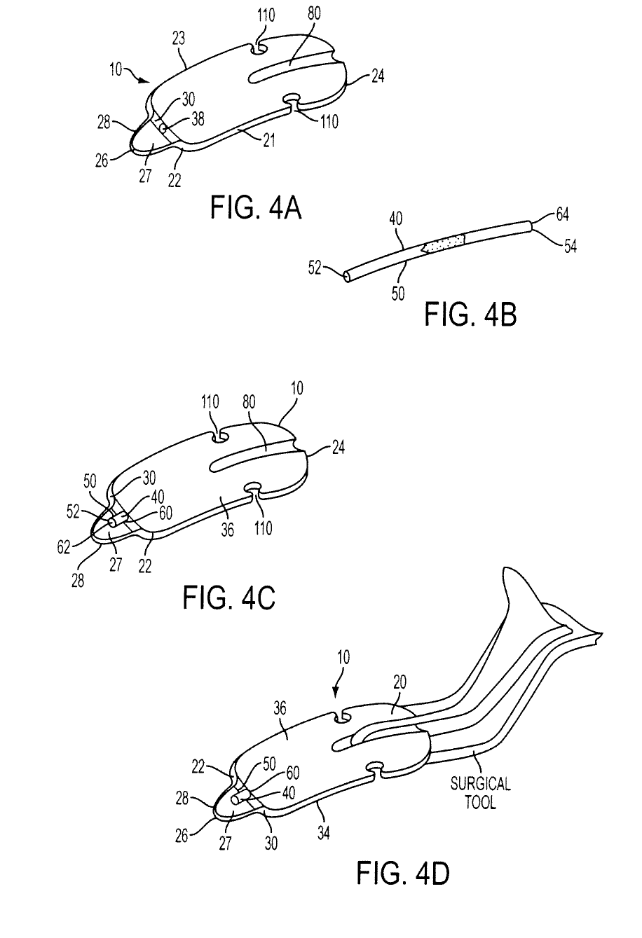

The conduit 40 of various embodiments, may include a first end 42 and a spaced second end 44. In the example shown, a portion of the conduit may be defined on a portion of a top surface 27 or within the insertion head 26 with the remaining portion defined within the elongate body 20 and extending from the forward end 22 to the back end 24 thereof. In some embodiments, the first end 42 of the conduit 40 may be located at the shearing edge 28 of the insertion head 26. The conduit 40 may be tapered and configured to be received by the insertion head 26. Alternatively, the first end 42 of the conduit 40 may be spaced from the shearing edge 28 and spaced from the shoulder surface 30 of the body 20. In an example, the first end 42 of the conduit 40 may be positioned at an acute angle with respect to the top surface 27 of the insertion head 26. In the example shown in FIG. 3A, the conduit may be formed integrally with the elongate body. One will appreciate however, and as shown in FIGS. 4A-4C, that the conduit 40 may also be a separate member which may be connected to the elongate body.

Referring to FIGS. 4A to 4C, in some embodiments, the conduit 40 may include an elongate tube 50 having a first end 52 and a spaced second end 54 which is integrated into the elongated body 20 to prepare the shunt. In some embodiments, the first end 52 to the second end 54 of the conduit 40 may be tapered to receive an obturator (as defined below). In other embodiments, the conduit may be a straight channel, and in still other embodiments, the conduit may be formed in other useful configurations. A longitudinally extending bore 38 may extend through the elongate body 20. In such embodiments, a proximal end of the bore may be defined in the forward end 22 of the elongated body 20 and may be positioned adjacent the top surface 27 of the insertion head 26. In use, at least a portion of the tube 50 may be positioned within the bore 38 of the body 20 such that the second end 54 of the tube is positioned proximate a distal end of the bore. Further, the first end 52 of the tube may extend through the proximal end of the bore and overlay a portion of the top surface 27 of the insertion head 26. In the example shown, the first end 52 of the tube may be spaced from both the shearing edge and the shoulder surface of the body 20. Alternatively, the first end 52 of the tube may be located at the shearing edge 28 (not shown). As one will appreciate, the tube 50 positioned within the bore of the body forms the “conduit” 40 described in reference to FIGS. 3A-3C.

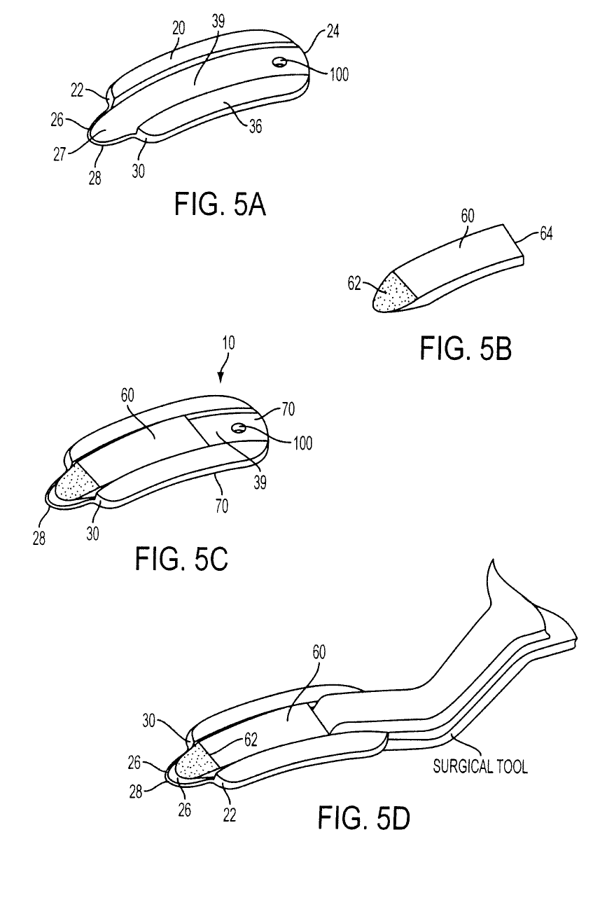

Turning to FIGS. 5A-5C, an alternative embodiment of the device is shown. Here, a longitudinally extending slit 39 may be defined on an upper surface 36 of the elongate body 20. In one exemplary embodiment, the slit 39 may extend from the forward end 22 to the back end 24 of the elongated body 20. In this embodiment, the wicking member 60 may be constructed and arranged such that the flow of aqueous humor from the inlet end 62 to the outlet end 64 may be regulated and aqueous humor enters the inlet end exits the outlet end. The wicking member 60 may be positioned within at least a portion of the slit of the body and overlay a portion of the top surface 27 of the insertion head 26. The inlet end 62 of the wicking member may be located at the shearing edge 28 or may be spaced from the shearing edge 28 of the insertion head 26, and in one example, the inlet end may be positioned at an acute angle with respect to the top surface of the insertion head.

Referring now to FIGS. 3A, 4C, and 5C, the elongate body 20 provides a means for grasping the body by a surgical tool such as, for example, forceps and the like. In one example, as shown in FIG. 5C, at least one planar surface 70 constructed and arranged for grasping by the surgical tool may be defined on at least a portion of at least one of the respective upper and lower surfaces of the elongate body. In this example, a portion of the slit in the elongate body forms one planar surface.

Alternatively, as shown in FIGS. 3A and 4C, the elongate body 20 may define on or more longitudinally extending grooves 80 in the exterior surface of the body, extending from the back end of the body which may be constructed and arranged for grasping by the surgical tool. One will appreciate that the groove 80 may be positioned in the upper surface 36 or in the lower surface 34 of the elongated body 20. Alternatively, a second longitudinally extending groove or a planar surface may be defined in the opposite spaced respective upper or lower surface to facilitate secure grasping of the device. As one will appreciate, any combination of planar surfaces and/or grooves on the respective upper and lower surfaces may be used to provide suitable grasping surfaces for the surgical tool.

After implantation, the shunt may be fixed to a portion of the sclera of the eye. For example, in the embodiment shown in FIG. 5C, the shunt may have at least one stitching loop or notch 100 defined in the elongate body. Sutures may be passed through the loop and secured to the sclera. In the example shown in FIG. 4C, the elongate body has a pair of spaced notches 110 that are constructed and arranged for facilitating suturing of the elongate body to eye tissue. Here, one notch of the pair of spaced notches is defined in each respective elongate edge 21 and 23 of the elongated body 20. Further, each notch 110 may have a keyhole shape. In another example shown in FIG. 3A, the elongated body 20 may have at least a pair of spaced bores or suture holes 120 extending between the upper and lower surfaces of the body. As one will appreciate, a suture may be passed through the bores for subsequent securing to the sclera. To simplify the surgical procedure, in some embodiments, at least one suture may be preloaded into the stitching loop, notches, bores, and the like of the device prior to inserting the device into the eye.

The stitching loops, notches, bores and such may be positioned at any location on the elongated body. However, in certain embodiments, the loops, notches or bores may be positioned a substantial distance from the back end 24 of the elongated body 20. For example, in some embodiments, the loop, notches or bores may be position at least about 2 mm from the back end of the elongated body, and in other embodiments, the loop, notches or bores may be positioned between 4 mm and 2.5 mm from the back end of the elongated body. In still other embodiments, the loop, notches or bores may be positioned 3 mm from the back end of the elongated body. Without wishing to be bound by theory, the position of the loop, notches or bores may reduce the incidence of, for example, fibrous by removing the sutures for attaching device to the eye from sutures necessary for closing the incision. For example, in one embodiment, the device may be placed in the eye such that the incision in the eye is about 2 to about 2.5 mm from the back end of the elongated body. Thus, the sutures associated with the loop, notches or bores are separated from the incision by about 0.5 mm to about 1.5 mm.

In one aspect, a wicking element, or valve may be employed to control the flow of aqueous from the anterior chamber to the suprachoroidal space, a hollow or empty conduit can act as a flow restrictor if properly sized. It is also contemplated that proper sizing of the conduit may be unnecessary as the flow may be limited by the absorptive capacity of the connective tissue surrounding the implanted device.

In a further aspect, it is contemplated that the absorptive capacity of the tissue surrounding the implant can be influenced by the choice of biomaterials from which the device may be made, or further influenced by coating the device, such as with, for example and not meant to be limiting, hyaluron, heparin, phosphorylcholine, butylmethacrylate, to encourage an aqueous boundary layer between the implant and host tissue. In this aspect, the absorptive capacity of the tissue surrounding the device may be further influenced by surface area. For example, within a fixed volume constraint, surface area may be enlarged by geometrical features such as fins, scales, fingers, corrugations, and texture.

In an alternative embodiment, the back end 24 of the shunt 10 may include a flattened, flexible tube which is configured to open when the anterior chamber pressure has risen to a level sufficient to cause the tube to open. In this aspect, the tube may be impermeable, permeable, or semi-permeable to aqueous fluid. In another aspect, the tube may be perforated with a plurality of holes or slots that are in fluid communication with the interior lumen of the tube. In yet another aspect, the posterior portion of the tube may be slit to create a plurality of capillary-like filaments. In another aspect, the posterior section of the tube may terminate in a plurality of filaments, wires, or hollow tubes that are configured for achieving aqueous flow through the hollow tubes or in the spaces between the filaments or wires. It is contemplated that the filaments, wires, or hollow tubes may move relative to each other and against each other and may be self-cleaning in the process. In another aspect, if the tube(s) is constructed from a permeable or semi-permeable material, the end of the tube(s) may be sealed such that the aqueous fluid flow is directed through the material of the tube(s). Thus, if the rate of fluid flow through the permeable or semi-permeable material is know, the interior surface areas of the closed tubes may be regulated to provide a certain combined rate of fluid flow.

In an alternative embodiment, the shunt may include a leaflet valve positioned within the conduit to effect regulation of the flow of fluid through the conduit. In one exemplary aspect, the leaflet valve may be positioned proximate the back end of the shunt, i.e., proximate the second end of the conduit.

In yet another embodiment, the conduit(s) formed therein the shunt may be formed with an initial width that may be modified by an intervention procedure after implantation of the shunt therein the patient. In one exemplary embodiment, a conventional laser may be used to size the conduit(s) therein the shunt.

In a further embodiment, the second end of the conduit of the shunt may be positioned to abut or otherwise connect with a biocompatible element. In exemplary aspects, the biocompatible element may be absorbent and may be disc-shaped or irregular in configuration. Of course, other geometrical shapes are also contemplated. In another aspect, portions of the biocompatible element may be formed from impermeable, permeable, or semi-permeable material that may be shaped as a membrane, collection of fibers, or perforated sheet-like material. For example, the surface shape of the biocompatible element can have geometrical features such as fins, scales, fingers, corrugations, and texture to increase the surface area of the biocompatible element thereby providing more exposure to adjacent tissues to increase the absorptive capacity of the shunt.

In an additional embodiment, the second end of the conduit may terminate in a broadened outflow path. In this aspect, the outflow path may be positioned in free fluid communication with the suprachoroidal space or may communicate with a hydrogel, hydrocolloid, or other absorbent material.

In another aspect, the conduit may be defined a posteriorly located reservoir that is substantially or open in part against the choroid when it is operatively positioned within the eye. In this aspect, when the ocular pressure is sufficiently elevated, the choroid is deflected and allows fluid to pass from the reservoir and into the suprachoroidal space. In an alternative embodiment, the reservoir may further include a flexible valve proximate the second end of the conduit. Here, when the ocular pressure is sufficiently elevated, the valve may be configured to open to allow fluid to exit the reservoir in the shunt to the suprachoroidal space.

In a further embodiment, the shunt may include a plurality of drainage holes on all of some or some of its surfaces that is in fluid communication with a central lumen or lumens. In one aspect, these holes may be used in combination with a recessed flow path such that the apposing tissue does not occlude the flow path.

In another embodiment, the shunt may include an interface between the implant and surrounding tissue of the suprachoroidal space that may be configured to act as a valve. In this aspect, when the pressure of the aqueous fluid within the single-lumen, multi-lumen, and/or perforated device may be sufficiently high, the single-lumen, multi-lumen, and/or perforated device expand to separate the apposing tissues and thereby expand the suprachoroidal space, which allows fluid egress from the shunt into the suprachoroidal space.

In another embodiment, the shunt may include a coiled spring that may be mounted proximate the second end of the conduit. In this aspect, the coils of the spring may be configured to move relative to each other and against each other. The coils may be self-cleaning in the process. The coils allow the passage of fluid between them and out of the second end of the conduit.

In various embodiments, the shunt may be made from any biological inert and biocompatible materials. The elongate body may be substantially rigid or may be substantially resilient and semi-rigid. Further, an exterior surface of the elongate body may be non-porous. Various medically suitable acrylics and other plastics known and utilized in the art may be used. The finish of the device may be to the standard for ophthalmic devices and should not create irritation to surrounding tissue. In one example, the device may be made by conventional liquid injection molding or transfer molding process.

In another exemplary embodiment, the shunt may be composed of a metal, ceramic, or polymeric material that can be coated with a polymeric material(s), which may prevent and or retard the attachment of cells and/or proteins present in the suprachoroidal space. In a further aspect, at least a portion of the shunt may be selectively coated to encourage cellular attachment to its external surface in some areas while discouraging it in others. It is also contemplated that at least a portion the conduit of the shunt also may be coated with a polymeric material(s) that may retard and/or the attachment of cells and/or proteins present in the aqueous fluid. The exemplified shunt may be a single piece or may be comprised of two or more parts to facilitate coating, and/or manufacturing, and/or assembly.

In another aspect, the shunt can incorporate therapeutic agent(s) for the reduction of intraocular pressure and/or prevention of fibrosis surrounding the inserted glaucoma drainage device. In one exemplary aspect, the shunt may include one or more agent(s) to reduce intraocular pressure said agent including: beta-blockers; alpha adrenergic agonists, prostaglandin analogs, carbonic anhydrase inhibitors, cholinesterase inhibitors, and combinations thereof. In one aspect the agent(s) may be released locally from the shunt at a controlled rate and amount into the suprachoroidal space. In another exemplary aspect, the shunt can comprise one or more anti-fibrosis agents that can be released rate locally from said glaucoma drainage device into the surrounding suprachoroidal space at a controlled rate and amount.

It is also contemplated that the shunt may include one or more anti-inflammatory agents, immunosuppressive agents, and/or anti-proliferate agents. The respective agents may be released rate locally from said glaucoma drainage device into the surrounding suprachoroidal space at a controlled rate and amount.

In a further embodiment, the shunt may incorporate additional components for securing the shunt in pace besides loops, notches or bores for suturing. For example, in one embodiment an adhesive may be applied to one surface of the shunt such as, for example, the top surface, which may bond to tissue surrounding the shunt securing the shunt in pace. The adhesive may have a removable backing that covers the adhesive during installation, and once the shunt is installed, the backing may be removed thereby exposing the adhesive.

In another embodiment, a small, flexible hair-like structures that are flexible in one direction, and rigid in another may protrude from the shunt. For example, the hair-like structures may bend as the shunt is inserted and remain rigid when the shunt is backed out of the incision thereby securing the shunt against surrounding tissue.

In still another embodiments, a securing feature may be added to the insertion head. For example, one or more barbs may be formed on the insertion head that allow the insertion head to enter tissue, but prevent the insertion head, and therefore the shunt itself, from backing out of the tissue. Such barbs may fold against the insertion head during insertion, and springing out to prevent the insertion head from backing out of the tissue. In additional embodiments, barbs may be placed on surfaces other than those of the insertion head of the shunt to prevent movement of the shunt once inserted. For example, in one embodiment barbs may be placed over an entire surface of the elongated body, and in another embodiment, one or more barbs may be used alone or in conjuction with the bores to secure the shunt in place.

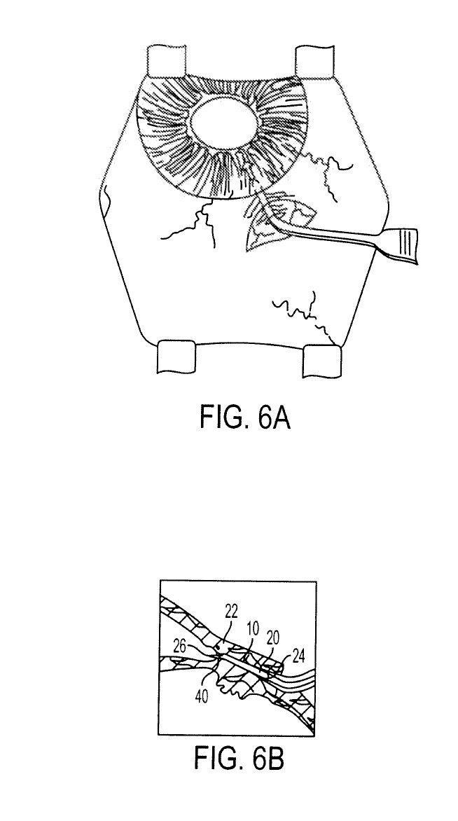

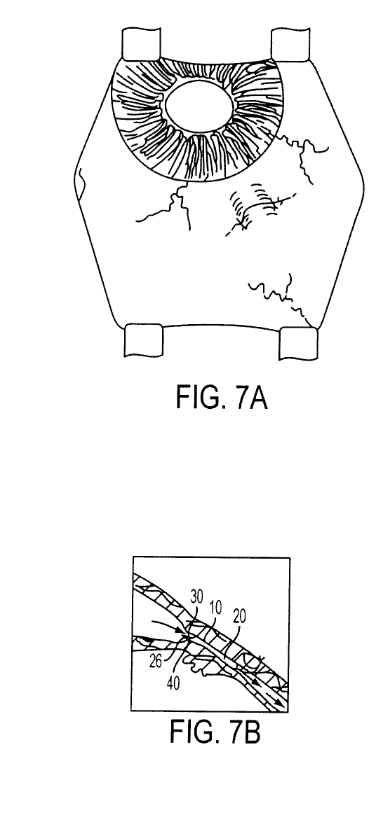

Turning now to FIGS. 6A-7B, the surgical method for implanting the device of the present invention into an eye will be explained. A first incision or slit may be made through the conjunctiva and the sclera at a location rearward of the limbus, that is, posterior to the region of the sclera at which the opaque white sclera starts to become clear cornea. Preferably, the first incision may be made about 3 mm posterior to the limbus. Also, the first incision may be made slightly larger than the width of the implant device. A conventional cyclodialysis spatula may be inserted through the first incision into the supraciliary space to confirm correct anatomic position.

A portion of the upper and lower surfaces of the shunt 10 proximate the back end of the body may be then grasped securely by the surgical tool, for example, a forceps, so that the forward end of the shunt may be oriented properly. In one example, the shunt may be oriented with the longitudinal axis of the device being substantially co-axial to the longitudinal axis of the grasping end of the surgical tool. The shunt 10 may be then disposed through the first incision and into the supraciliary space of the eye. The shearing edge of the shunt may be advanced anteriorly in the supraciliary space and inserted into and through the anterior chamber angle of the eye. More particularly, the shearing edge of the insertion head may pass between the scleral spur and the ciliary body posterior to the trabecular meshwork. The shunt may be continually advanced anteriorly until a portion of the insertion head and the first end of the conduit is disposed within the anterior chamber of the eye. Thus, the first end of the conduit may be placed into fluid communication with the anterior chamber of the eye. The back end of the elongate body may be disposed into the suprachoroidal space of the eye so that the second end of the conduit may be placed into fluid communication with the suprachoroidal space.

Preferably the back end of the elongate body may be positioned under the posterior margin/lip of the scleral incision site to mitigate the risk of obstruction due to fibrosis or other tissue reactions associated with surgical wound healing. The placement of the back end of the elongate body several millimeters posterior to the surgical incision is preferably done in a manner that is atraumatic to the sclera and choroid that border the suprachoroidal space. Additionally placement of the suture that anchors the shunt within the suprachoroidal space is preferably anterior to the surgical incision site. Accordingly, this allows the back end of the elongate body to be located at a far distance from fibrosis or other tissue reactions that could occur at the incision cite and therefore result in blockage of aqueous humor outflow into the suprachoridal space.

The shoulder surface of the forward end of the shunt may be seated proximate an interior surface of the supraciliary space and may not be introduced into the anterior chamber. The shoulder surface may aid in forming a tight seal to prevent leakage of aqueous humor around the device as well as helping to prevent unwanted further anterior movement of the shunt. The shape of the cleft formed by the insertion head forms a tight seal about the exterior surface of the body, and, if used, the fusiform cross-sectional shape of the body prevents gaping of the formed cleft on either elongate edge of the shunt.

The shunt may be then secured to a portion of the sclera to aid in fixating the shunt. The first incision is subsequently sutured closed. As one will appreciate, the suture used to fixate the shunt may also be used to close the first incision.

It will be seen that upon implantation, the drainage device forms a cyclodialysis with the conduit providing transverse communication of aqueous humor through the shunt along its length. Aqueous humor thus delivered to the suprachoroidal space will then be absorbed therein, and additional reduction in pressure within the eye is to be expected.

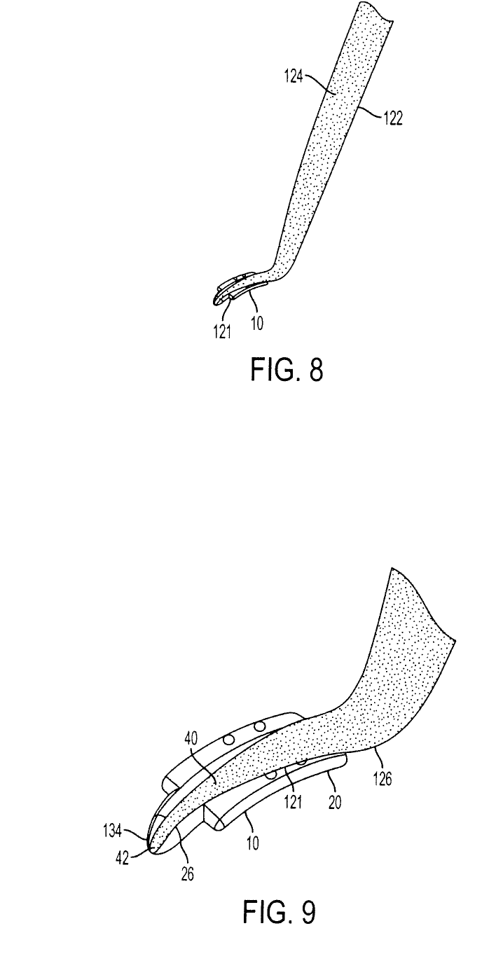

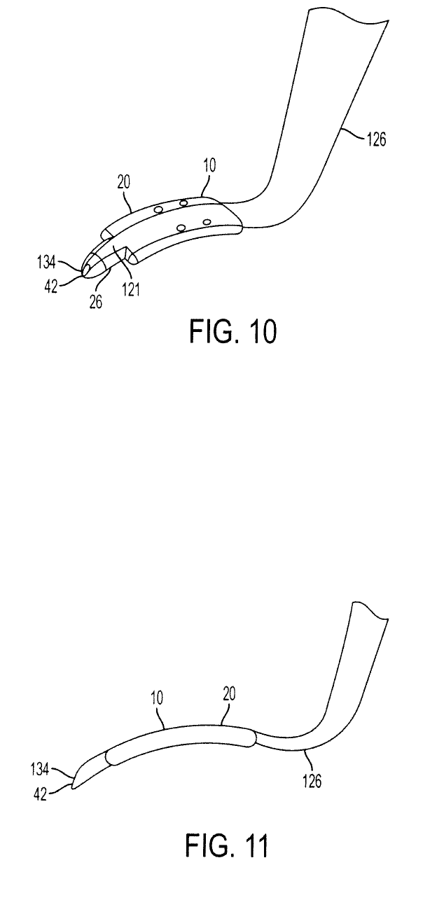

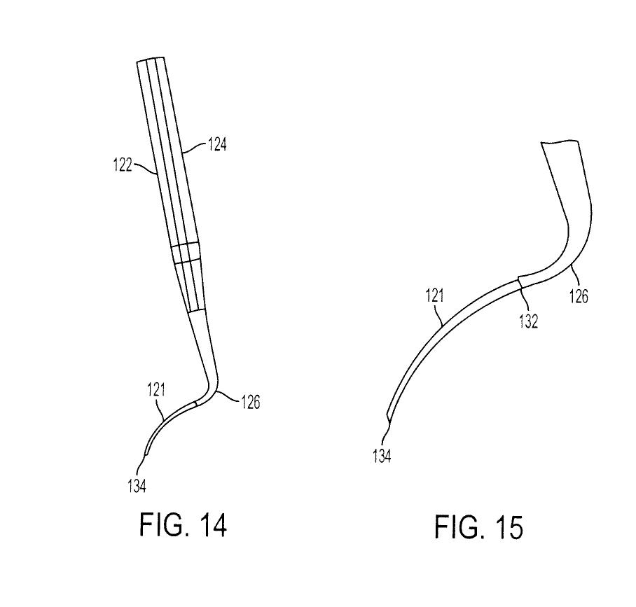

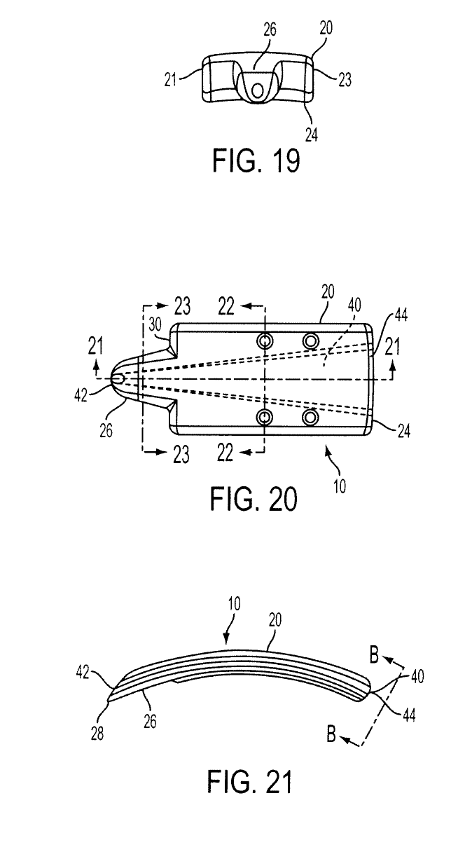

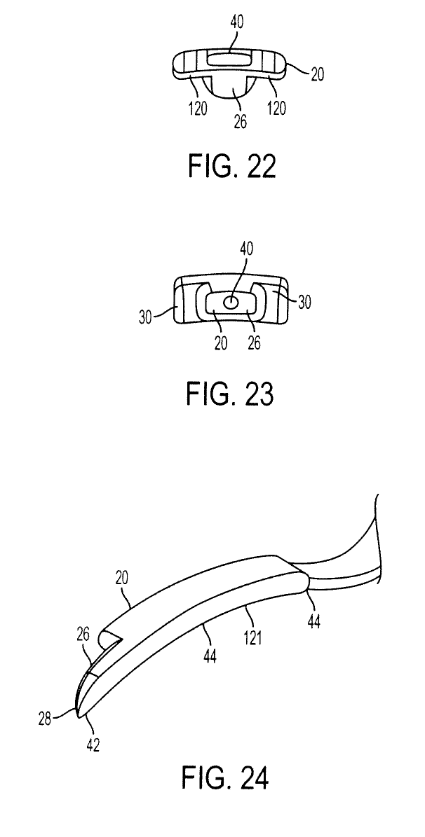

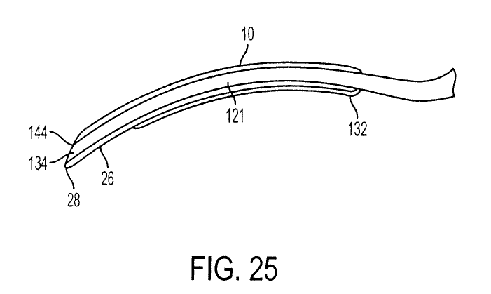

In another embodiment of the invention is directed to an ophthalmic shunt assembly including a shunt, such as, the shunt, described herein above and an obturator 121. As disclosed in the FIGS. 8-25, an obturator 121 or “stylet” may be removeably positioned within at least a portion of the interior of the conduit 40 thereby filling the at least a portion of interior volume of the conduit to prevent the conduit 20 of the shunt 10 from becoming obstructed as the shunt is advanced into place. For example, in some embodiments, the obturator may be positioned to fill the entire conduit, such that both the first end 42 of the conduit and the opening at the back end 24 of the elongated body 20 are completely filled by the obturator. In some such embodiments, the obturator may be flush with the opening of the first end of the conduit, or in other embodiments, the obtrutor may extend beyond and protrude from the first end of the conduit. Therefore, the obturator 121 may be configured to block the first end 42 of the conduit and may prevent accumulation of tissue and blockage of the conduit that could otherwise be forced into the first end of the conduit as the insertion head is forcefully pressed though the eye tissue.

In a further aspect, the obturator may provide a means for “priming” the conduit. In such embodiments, fluid may displace air or the material of the obturator 121 as it is removed from the conduit 40.

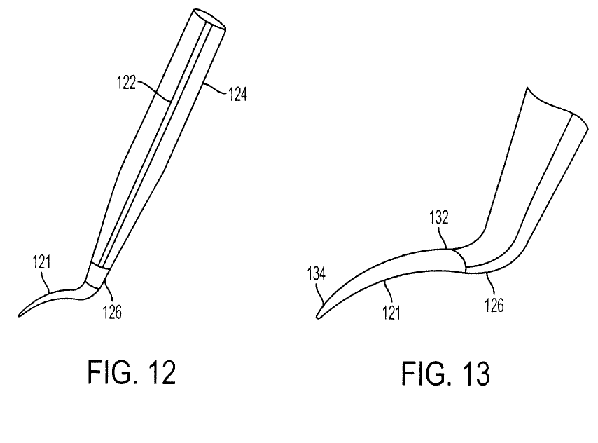

In another aspect, the obturator 121 may be configured to act as the insertion instrument itself and obviate the need to grasp the device on its outside surfaces or surface features. For example, referring to FIGS. 12-15, an exemplary embodiment the obturator 121 may include a handle portion 122. The handle portion 122 of some embodiments may be integral with obturator such that the handle is formed from the same material as the obturator. In such embodiments, the obturator may make up a mount portion of the device. In other embodiments, the handle portion 122 may be removable attached to the obturator. The handle portion 122 may have a proximal end portion 124 and a distal end portion 126. The distal end portion 126 may be ergonomically designed to orient the hand of the surgeon, upon his or her employment of the obturator 121, in a naturally functional position. The proximal end portion 124 may be designed to facilitate proper placement of shunt. For example, the proximal end may be angled or curved such that the shunt is properly or conveniently aligned when the operator grasps the distal end portion 126. In one embodiment, the proximal end portion may extend along a longitudinal axis, and the distal end portion is oriented relative to the longitudinal axis of the proximal end portion at an angle, for example, between 90 and 150 degrees. However, it will be appreciated that angles outside of this range may be necessary, and may be employed by one skilled in the art which may or may not maintain the ergonomic character of the handle. Further, the union of the proximal end portion and distal end portion is preferably rounded and or smooth to avoid sharp edges which could cause injury to surrounding tissues upon insertion of the shunt.

The obturator 121 may be configured to create a temporary, selectively releasable, engagement with the means for mounting provided by the elongate body of the shunt. Referring to FIGS. 8-11 and 24-25, one example of the operative engagement between the obturator 121 and the shunt 10 is shown. In one aspect, to achieve the desired engagement, the obturator 121 may have a first end 134 and a second end 132, wherein the first end 134 may be connected to the distal end portion 126 of the handle, and extends outwardly toward to the second end 132. At least a portion of the second end 132 may be configured for operative receipt by the conduit 40 in the shunt, such that the shunt may be selectively fixed to the second end of the obturator, which ensures that movement of the second end of the obturator 121 may cause the same relative movement of the mounted shunt 10. IN certain embodiments, the first end 134 may be flush with the distal end of the conduit thereby blocking the distal opening of the conduit.

In one aspect, at least a portion of the mount portion may be selectively withdrawn within a portion of the distal end portion of the handle. It is further contemplated that the distal end portion of the handle can define a stop that may be configured to prevent the rearward movement of the shunt as the mount portion is withdrawn from the distal end portion of the handle.

In some embodiments, at least a portion of the second end 134 of the obturator has a shape that closely conforms to a portion of the interior of the conduit. For example, in one embodiment, the conduit has a wedge shape such that the width of the conduit decreases from back to front. Complementarily, at least a portion of the mount portion of the obturator has a wedge shape such that the width of the mount portion accordingly decreases moving longitudinally from the first end to the second end.

In another embodiment, the second end of the mounting portion can be configured to effectively block the first end of the conduit 40. In this aspect, the obturator 121 forms a shoulder surface 140 that is configured to operatively engage the back end 24 of the body of the shunt. This allows a pushing force to be applied to the back end of the shunt. In another embodiment, the obturator 121 may define a plurality of tabs 142 that are connected to edge portions of the shoulder surface and that extend outwardly away from the shoulder surface. In this example, a plurality of male tabs 142 may define a notch 144 that is configured to make releasable contact portions of the exterior surface of the shunt 20 proximate the back end 24 of the shunt. This would allow for control over the orientation of the shunt 10 as it is mounted onto the obturator and would insure that movement of the second end of the obturator 121 causes the same relative movement of the mounted shunt 10.

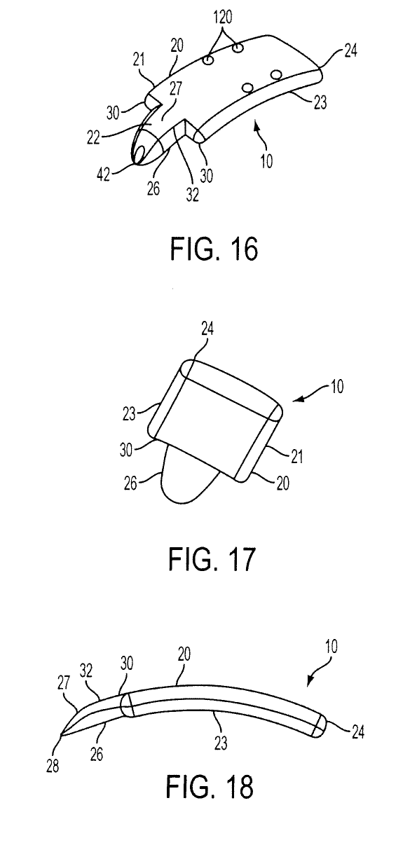

Referring to FIGS. 16-23, an exemplary embodiment of a shunt 10 is shown. In this embodiment, the shunt 10 includes elongate body 20 and conduit 40. The elongate body may have a forward end 22, a spaced back end 24, and extends along a longitudinal axis. The body may also have an insertion head 26 that extends generally longitudinally from the forward end thereof. The elongate body further has a first elongate edge 21 and a second elongate edge 23 that extend respectively from the forward end to the back end of the body. The insertion head is adapted for insertion into the anterior chamber of the eye and defines a shearing edge 28 configured for cutting eye tissue engaged thereby. In the example shown, the shearing edge of the insertion head may have an arcuate shape. However, as one skilled in the art will appreciate, other shapes, such as, for example, chisel shapes, scalpel shapes, and the like, are contemplated for the shearing edge.

The juncture of the insertion head 26 against the forward end 22 of the body defines at least one shoulder surface 30 thereon. In one example, the insertion head has a base portion 32 having a first width and where the respective first and second elongate edges are spaced apart a second width that is greater than the first width. The shoulder surface 30 of the body may be adapted to engage tissue portions of the anterior chamber angle of the eye that are adjacent an interior surface of the interior chamber. The shoulder surface 30 also aids in limiting the anterior movement or displacement of the device when implanted, which helps prevent the forward end 22 of the drainage device from penetrating and entering the anterior chamber. In the example illustrates in FIGS. 16-23, the elongate body has an upper surface and a spaced lower surface, and a cross-sectional portion of the insertion head has a substantially constant thickness. In this example, the insertion head has a base portion, and a portion of the insertion head may extend outwardly from the base portion and have substantially uniform thickness.

Further, the insertion head 26 can have, in one exemplary embodiment, a shape that acts to dilate tissue as it is inserted into position. This may cause the tissue to stretch around the exterior surface of the insertion head such that the insertion head is self-sealing. In another example, a portion of the insertion head, spaced from the shearing edge, may define a circumferentially extending groove or waist that is configured such that the stretched tissue can relax fractionally to both seal and fixate the shunt relative to the insertion.

The body 20 has a length from the forward end to the back end of such extent to extend from proximate the interior surface of the anterior chamber to the suprachoroidal space of the eye. The back end 24 of the body is adapted for insertion within the suprachoroidal space of the eye. Along at least a portion of its length, the body may be substantially planar or may have an arcuate shape that is adapted to extend along a portion of the curvature of the sclera of the eye. As one will appreciate from the illustrated embodiment, the body is generally thin to provide a less irritating fit within the eye.

In one example, the elongate body 20 has a substantially fusiform cross-sectional shape. This fusiform shape aids in stabilizing the device when implanted as tissues of the anterior chamber angle surround portions of the exterior surface of the body. A variety of cross-sectional shapes are contemplated for the elongate body as long as a shoulder surface is defined in the forward end. It is also contemplated that at least a portion of the elongate body has a substantially fusiform cross-sectional shape. In yet another aspect, the body 20 can have an arcuate shape over at least a portion of its cross-sectional width that is configured to extend along a portion of the curvature of the eye.

In the exemplary shunt shown, the first end 42 of the conduit may be defined within a portion of a top surface 27 of the insertion head 26 and the second end 44 may be defined within a portion of the back end of the elongate body. The first end of the conduit may be positioned proximate the shearing edge of the insertion head and is spaced from the shoulder surface 30 of the body. In one exemplary aspect, at least a portion of the conduit may be configured for operative receipt of a distal or mounting end of a surgical tool.

In one example, the first end 42 of the conduit may be positioned at approximately the same angle as the adjacent portion of the top surface of the insertion head. In another example, the width of the conduit may gradually increase as the conduit extends longitudinally from the first end to the second end. In the example shown in FIG. 20, the conduit may be formed integrally with the elongate body. One will appreciate however, that the conduit 40 may also be a separate member which is connected to the elongate body.

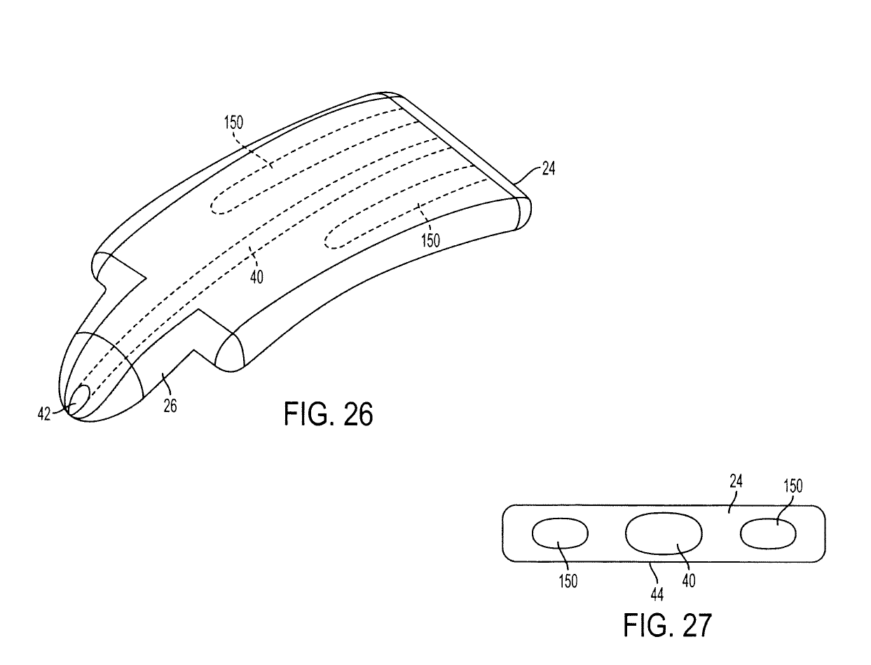

In a further aspect, the elongate body 20 may define at least one slot 150 that is configured for operative receipt of a distal end of a surgical tool. As illustrated in FIGS. 26 and 27, an alternative embodiment of the shunt 10 is shown including slots 150. In this embodiment, the obturator 121 may have at least a pair of prongs that are configured for selective and releasable mounting of the shunt thereto. In one aspect, the obturator 121 may include a first and second prong that extend outwardly from the distal end portion of the handle. The respective first and second prongs may be configured to be operatively received into corresponding slots 150 that are defined in the shunt.

In one aspect, the first and second prongs of the obturator 121 and the slots 150 of the shunt may be configured such that upon insertion of the prongs into the slots, the shunt is positionally fixed with respect to the obturator. Thus, the shunt may be readily implantable as it resists twisting relative to and about the mounting portion of the obturator. In this aspect, the first and second prongs add additional support to the connection between the mount portion of the obturator and the shunt to decrease slippage and allow for more precise control of the shunt during implantation. It will be noted, however, that additional or fewer prongs may be utilized as the situation requires, and that the inclusion of an embodiment having a plurality of prongs is merely for illustrative purposes and is not meant to be limiting. Further, substitute prong cross-sectional geometric shapes, such as half circle, triangular, and the like are also contemplated.

Additional prongs may be formed in the mount portion of the obturator that may be configured to be operatively received into the conduit. In this aspect, the additional prong performs substantially the same function as the prong in the single pronged embodiment that is described above.

After implantation the shunt may be fixed to a portion of the sclera of the eye. To facilitate fixation, the shunt 10 may have at least one spaced bore 120 that extends between the upper and lower surfaces of the body 20. As one will appreciate, a suture can be passed through the bores for subsequent securing to the sclera. To simplify the surgical procedure, at least one suture may be preloaded into the bores of the device prior to inserting the device into the eye. However, it should be noted that multiple bore arrangements may be used for suturing the device. For example, multiple sets of bores may be provided, thereby providing multiple possible locations for suturing the device dependant on the application, providing additional flexibility.

The surgical method for implanting the device of the present invention into an eye will be explained. A first incision or slit is made through the conjunctiva and the sclera at a location rearward of the limbus, that is, posterior to the region of the sclera at which the opaque white sclera starts to become clear cornea. Preferably, the first incision is made about 3 mm posterior to the limbus. Also, the first incision is made slightly larger than the width of the implant device. A conventional cyclodialysis spatula may be inserted through the first incision into the supraciliary space to confirm correct anatomic position.

The obturator 121 maybe inserted into the shunt so that the shunt is oriented properly. As discussed above, the obturator may penetrate the conduit, or include additional prongs for holding the shunt in position. By manipulation of the obturator, the shunt 10 is then disposed through the first incision and into the supraciliary space of the eye. The shearing edge of the shunt may then be advanced anteriorly in the supraciliary space and may be inserted into and through the anterior chamber angle of the eye. More particularly, the shearing edge of the insertion head may pass between the scleral spur and the ciliary body posterior to the trabecular meshwork. The shunt may be continually advanced anteriorly until a portion of the insertion head and the first end of the conduit is disposed within the anterior chamber of the eye. The tissue surrounding the incision can be stretched about the exterior of the insertion head to substantially form a fluid seal or water-tight seal about the insertion head (at the junction between the suprachoroidal space and the anterior chamber). Thus, the first end of the conduit is placed into fluid communication with the anterior chamber of the eye. Following removal of the obturator, the back end of the elongate body may be disposed into the suprachoroidal space of the eye so that the second end of the conduit is placed into fluid communication with the suprachoroidal space.

In one aspect, the obturator may allow for a less traumatic device introduction and placement than other available surgical methods. In one exemplified aspect, the obturator may preclude obstruction of the conduit. As shown in the figures, the obturator 121 may be removably positioned within at least a portion of the conduit, thereby filing at least a portion of the interior volume of the conduit proximate the first end of the conduit and preventing obstruction of the first end of the conduit. Thus, in one aspect, the obturator can be configured to selectively block the first end of the conduit to prevent any accumulation of tissue that could cause partial or full blockage of the conduit. Once the shunt is installed, removal of the obturator from the conduit may result in an aspiration of fluid into the conduit, thereby establishing a fluid flow through the conduit from the anterior chamber into the suprachoroidal space.

In another aspect, it is contemplated that second end 134 of the obturator 121 can be configured to extend outwardly beyond the exterior surface of the insertion head. In this aspect, at least a portion of the second end of the obturator can define a shearing edge that is configured for penetrating tissue. In this aspect, the shearing edge can be used as a dilator or instrument for dissection.

In use, the shoulder surface of the forward end of the shunt may be seated proximate an interior surface of the supraciliary space and is not introduced into the anterior chamber. The insertion head and the shoulder surface complementarily aids in forming a tight seal to prevent leakage of aqueous humor around the device as well as helping to prevent unwanted further anterior movement of the shunt. The shape of the cleft formed by the insertion head forms a tight seal about the exterior surface of the body, and, if used, the fusiform cross-sectional shape of the body may prevent gaping of the formed cleft on either elongate edge of the shunt.

The shunt may then be sutured to a portion of the sclera to aid in fixating the shunt. The first incision is subsequently sutured closed. As one will appreciate, the suture used to fix the shunt may also be used to close the first incision. In a further aspect, the conduit of the shunt may be primed by withdrawing the obturator from the conduit, which aspirates fluid into the conduit while displacing the material of the obturator.

It will be seen that upon implantation, the drainage device can form a cyclodialysis with the conduit providing transverse communication of aqueous humor through the shunt along its length. Aqueous humor thus delivered to the suprachoroidal space will then be absorbed therein, and additional reduction in pressure within the eye is to be expected.

### EXAMPLE

After making a conjuctival incision, a scleral incision is made approximately 5 mm from the limbus and approximately 5 mm wide. Before inserting the shunt into the suprachoroidal space through the scleral incision, a double-armed 10-0 prolene suture is passed from the under surface to the top side of the shunt through the two bores, which are approximately 3 mm from the distal end of the shunt. Once the shunt is in proper position, for example, with the proximal end approximately 3 mm into the anterior chamber and the shoulder of the shunt against the adhesion between the suprachoroidal space and anterior chamber, the two suture needles are passed through the anterior lip of the scleral incision, and the suture is pulled tight and tied, locking the shunt in position. The posterior lip of the incision is then lifted up with forceps, and the distal end of the shunt is tucked under the posterior lip, placing it approximately 2.5 mm posterior to the scleral incision. The posterior placement keeps the distal lumen well away from the scleral incision, where fibrous (scar) tissue could potentially obstruct the lumen. The scleral incision is then closed with one or more 10-0 prolene or nylon sutures.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An ophthalmic shunt implantable in an eye, comprising:
an elongate body having a forward end, a back end, and a tapered insertion head extending from the forward end of the elongate body, the insertion head defining a shearing edge constructed and arranged for cutting eye tissue engaged thereby, the forward end and the insertion head of said body further defining a shoulder surface; and
a conduit having a first end defined at the shearing edge of said insertion head and extending through said body from the forward end to the back end thereof, the first;
wherein the elongate body is configured to position at least a portion of the insertion head and the first end of the conduit through an incision formed by the shearing edge of the insertion head and into fluid communication with the anterior chamber of the eye and to seat at least a portion of the insertion head against the incision.

2. The shunt of claim 1, wherein the elongate body has a substantially fusiform cross-sectional shape.

3. The shunt of claim 1, wherein the elongate body has a lower surface, and wherein a portion of the insertion head is substantially co-planar to the lower surface thereof.

4. The shunt of claim 1, wherein the elongate body has an arcuate shape along at least a portion of its length that is adapted to extend along the curvature of the sclera.

5. The shunt of claim 1, wherein the conduit is configured to receive at least a portion of an obtruator.

6. The shunt of claim 5, wherein a first end of the obturator is at least flush with the first end of the conduit.

7. The shunt of claim 1, wherein the first end of the conduit is positioned at an acute angle with respect to the insertion head.

8. The shunt of claim 1, wherein the elongate body has at least one suture hole configured to facilitate suturing the elongate body to eye tissue.

9. The shunt of claim 1, wherein the elongate body has a first elongate edge and a spaced second elongate edge, and wherein said body has at least a pair of spaced suture holes configured to facilitate suturing the elongate body to eye tissue, one suture hole of the pair of spaced suture holes being defined in each respective elongate edge.

10. The shunt of claim 1, wherein the elongate body has an upper surface and a spaced lower surface, and wherein the body has at least a pair of spaced suture holes extending between the upper and lower surfaces of said elongate body, the pair of spaced suture holes configured to facilitate suturing the elongate body to eye tissue.

11. An ophthalmic shunt assembly, comprising:
an elongate body having a forward end, a back end, and an insertion head extending from the forward end of the elongate body, the insertion head defining a shearing edge constructed and arranged for cutting eye tissue engaged thereby, the forward end and the insertion head of said body further defining a shoulder surface;
a conduit having a first end defined at the shearing edge of said insertion head and extending through said body from the forward end to the back end thereof; and
an obturator,
wherein the conduit is configured to receive at least a portion of the obturator, and
wherein the elongate body is configured to position at least a portion of the insertion head and the first end of the conduit through an incision formed by the shearing edge of the insertion head and into fluid communication with the anterior chamber of the eye and to seat at least a portion of the insertion head against the incision.

12. The shunt assembly of claim 12, wherein the elongate body has a substantially fusiform cross-sectional shape.

13. The shunt assembly of claim 12, wherein a first end of the obturator is at least flush with the first end of the conduit.

14. The shunt assembly of claim 12, wherein the obturator further comprises a handle located at an opposing end of the first end of the obturator.

15. The shunt assembly of claim 12, wherein the elongate body has at least one suture hole configured to facilitate suturing the elongate body to eye tissue.

16. The shunt assembly of claim 12, wherein the elongate body has a first elongate edge and a spaced second elongate edge, and wherein said body has at least a pair of suture holes configured to facilitate suturing the elongate body to eye tissue, one suture hole of the pair of suture holes being defined in each respective elongate edge.

17. The shunt assembly of claim 12, wherein the conduit comprises a valve.

18. The shunt assembly of claim 12, wherein a first end of the conduit is positioned at an acute angle with respect to the insertion head.

19. The shunt assembly of claim 12, wherein the conduit comprises at least a rigid front end.

20. The shunt assembly of claim 12, wherein the elongate body further comprises a flexible tube in fluid communication with the conduit to provide a channel for fluid flow from an anterior space to a suprachoroidal space.

21. The shunt assembly of claim 21, wherein a posterior end of the conduit comprises at least one of a plurality of filaments, wires or hollow structures.

22. The shunt assembly of claim 12, wherein the assembly delivers at least one therapeutic agent to a suprachoroidal space.

23. A method for treating glaucoma in an eye, comprising:
a. providing a biocompatible ophthalmic shunt, wherein the ophthalmic shunt comprises:
i. an elongate body having a forward end, a back end, and a tapered insertion head extending from the forward end of the elongate body, the insertion head defining a shearing edge constructed and arranged for cutting eye tissue engaged thereby, the forward end and the insertion head of said body further defining a shoulder surface; and
ii. a conduit having a first end defined at the shearing edge of said insertion head and extending through said body from the forward end to the back end thereof;
wherein the elongate body is configured to position at least a portion of the insertion head and the first end of the conduit through an incision formed by the shearing edge of the insertion head and into fluid communication with the anterior chamber of the eye and to seat at least a portion of the insertion head against the incision to seal the incision;
b. inserting at least a portion of the shearing edge of the insertion head of the shunt into and through an anterior chamber angle and into the anterior chamber of the eye, with the first end of the conduit into fluid communication with the anterior chamber of the eye;
c. introducing the insertion head anteriorally to seat the shoulder surface of the implant adjacent an interior surface of a supraciliary space of the eye;
d. disposing the back end of the elongate body of the shunt into a suprachoroidal space of the eye so that a second end of the conduit is in fluid communication with the suprachoroidal space;
e. securing the shunt to the eye.

24. The method of claim 24, further comprising, prior to the insertion of the insertion head into the anterior chamber making a first incision in and through the conjunctiva and the sclera at a position posterior to the limbus.

25. The method of claim 24, further comprising delivering at least one therapeutic agent to a suprachoroidal space.
